(19) United States
(12) Patent Application Publication
(10) Pub. No.: US 2020/0077897 A1
(43) Pub. Date: Mar. 12, 2020

(54) DEVICE AND METHOD FOR TOMOGRAPHICALLY VISUALIZING VISCOELASTICITY OF TISSUE

(71) Applicant: UNIVERSITY PUBLIC CORPORATION OSAKA, Osaka (JP)

(72) Inventor: Souichi SAEKI, Osaka (JP)

(21) Appl. No.: 16/685,094

(22) Filed: Nov. 15, 2019

Related U.S. Application Data
(63) Continuation of application No. PCT/JP2018/018455, filed on May 14, 2018.

(30) Foreign Application Priority Data
May 15, 2017 (JP) 2017-096134

(51) Int. Cl. A61B 5/00; G01B 9/02; G01N 29/06

(52) U.S. Cl. CPC A61B 5/0066; G01B 9/02091; A61B 5/004; A61B 5/442; G01N 29/0672

(57) ABSTRACT

A device according to an embodiment includes an optical system employing the OCT, and tomographically visualizes the viscoelasticity of tissue of an object. The device includes an optical mechanism that guides light from a light source to the tissue of the object to scan the tissue, a loading device that applies deformation energy to the tissue, a control computation unit that computes tomographic distribution of the viscoelasticity of the tissue by controlling driving of the loading device and the optical mechanism and processing an optical interference signal from the optical system on the basis of the driving, and a display device that displays the viscoelasticity of the tissue in a form of tomographic visualization. The loading device is a non-contact loading device that loads acoustic radiation pressure in the tissue by outputting ultrasonic waves toward a focus point set in the object.

### CROSS-REFERENCE TO RELATED APPLICATIONS

This application is a continuation application of International Application No. PCT/JP2018/018455, filed on May 14, 2018, which claims priority to and the benefit of Japanese Patent Application No. 2017-096134, filed on May 15, 2017. The contents of these applications are incorporated herein by reference in their entirety.

### BACKGROUND OF INVENTION

### 1. Field

The present invention relates to a device and a method capable of tomographically displaying in microscale the viscoelasticity of tissue of an object to be measured.

### 2. Description of Related Art

In recent years, for further development of medical diagnostic technology, clinical applications of optical coherence tomography (hereinafter referred to as “OCT”) are underway. The OCT is tomography using low-coherence optical interference, which enables visualization of distribution of biological tissue forms at high spatial resolution in microscale. In addition, the acquisition rate in two-dimensional OCT is not lower than the video rate, which also provides high temporal resolution.

In this regard, the present inventor and other researchers have proposed a technique for tomographically visualizing the mechanical characteristics of biological tissue by using the OCT (WO 2016/031697). This is based on an idea that quantitative evaluation of microbiomechanics in tissue contributes to improvement of diagnostic technology because a change in the mechanical characteristics of tissue appears more significantly than a change in the quantity of matrices in biological tissue of cartilage, skin, and the like. This technique aims at development of an OCT system capable of evaluating the characteristics (viscoelasticity) of biological tissue.

Related Art List

(1) WO 2016/031697

Note that, for evaluation of such mechanical characteristics, an algorithm that deforms biological tissue in OCT measurement is embedded. Thus, the technology of WO 2016/031697 employs a method of bringing a piezoelectric device into contact with a surface of an object to apply a load thereto. In a case where an object, such as regenerated tissue, requiring strict quality assurance is to be measured, however, the contact of such a piezoelectric device may cause tissue contamination, which may hinder practical use of the system. In view of the findings, the inventor has recognized the need to improve the OCT system.

### SUMMARY OF INVENTION

In view of the above and other circumstances, one of objects of the present invention is to provide a technology that enables mechanical characteristics evaluation through OCT measurement even on an object requiring strict quality assurance.

An embodiment of the present invention relates to a viscoelasticity visualizing device that includes an optical system employing the OCT, and tomographically visualizes the viscoelasticity of tissue of an object to be measured. The device includes an optical mechanism that guides light from a light source to the tissue of the object to scan the tissue, a loading device that applies deformation energy to the tissue, a control computation unit that computes tomographic distribution of the viscoelasticity of the tissue by controlling driving of the loading device and the optical mechanism and processing an optical interference signal from the optical system on the basis of the driving, and a display device that displays the viscoelasticity of the tissue in a form of tomographic visualization. The loading device is a non-contact loading device that loads exciting force caused by acoustic radiation pressure in the tissue by outputting ultrasonic waves toward a focus point set in the object.

Another embodiment of the present invention relates to a viscoelasticity visualizing method for tomographically visualizing viscoelasticity of tissue of an object to be measured. The method includes a loading step of applying a load in the tissue by acoustic radiation pressure of ultrasonic waves, an interference signal acquiring step of acquiring, by using the OCT, an optical interference signal based on backscattered light from the tissue that is deformed by the application of the load, a computing step of processing the optical interference signal and computing tomographic distribution of the viscoelasticity of the tissue on the basis of a change in a predetermined mechanical feature quantity accompanying deformation of the tissue, and a displaying step of tomographically visualizing the viscoelasticity of the tissue.

According to the present invention, a technology enabling mechanical characteristics evaluation through OCT measurement even on an object requiring quality assurance is provided.

### BRIEF DESCRIPTION OF DRAWINGS

FIG. 1 is a diagram schematically illustrating a configuration of a viscoelasticity visualizing device according to an embodiment;

FIGS. 2A and 2B are explanatory diagrams illustrating a method for applying load to an object in a non-contact manner;

FIGS. 3A to 3C schematically show procedures of a recursive cross-correlation method;

FIGS. 4A to 4C schematically show procedures of sub-pixel analysis;

FIG. 5 is a flowchart illustrating a flow of a process of tomographically visualizing viscoelasticity performed by a control computation unit;

FIG. 6 is a flowchart illustrating a real-time viscoelasticity computing process in S18 of FIG. 5 in detail;

FIG. 7 is a flowchart illustrating a tomographic viscoelasticity distribution computing process in S20 of FIG. 5 in detail;

FIG. 8 is a flowchart illustrating a viscoelasticity computation presenting process in S80 of FIG. 7 in detail;

FIGS. 9A and 9B illustrate results of experiment with samples that are imitations of biological tissue;

FIGS. 10A and 10B illustrate results of experiment with samples that are imitations of biological tissue;

FIGS. 11A and 11B illustrate results of experiment with a layered sample with rigidity varying by the layer;

FIGS. 12A and 12B illustrate results of experiment with a layered sample with rigidity varying by the layer;

FIGS. 13A to 13C illustrate results of experiment on viscosity evaluation; and

FIGS. 14A to 14C are diagrams illustrating load application methods according to modifications.

### DETAILED DESCRIPTION

One embodiment of the present invention is a viscoelasticity visualizing device using the OCT. The device includes a light source, an optical mechanism, a loading device, a control computation unit, and a display device, and tomographically visualizes the viscoelasticity of tissue of an object to be measured. An “object” may be biological tissue of skin, cartilage, etc., or regenerated tissue (tissue generated from cultured cells) of regenerated skin, regenerated cartilage, etc. The loading device outputs ultrasonic waves toward a focus set in an object, and loads acoustic radiation pressure in the tissue. The focus is a point in the object on which exciting force caused by the acoustic radiation pressure concentrates, and is set by the control computation unit.

In this embodiment, loading by the acoustic radiation pressure and detection by the OCT are performed for tomographic visualization of tissue viscoelasticity. The load applied by the acoustic radiation pressure may be a dynamic load (exciting force). The load is applied to a desired cross-sectional position in the object by the acoustic radiation pressure in a non-contact manner, and induces tissue deformation. In addition, the OCT enables non-contact and non-invasive tomographic measurement. Thus, according to the embodiment, both of the loading device and the optical mechanism are in no contact with the object. This prevents contamination of the object and enables evaluation of the mechanical characteristics (viscoelasticity) thereof even when the object requires strict quality assurance.

The axis of light emitted toward the object may be set to pass through the focus of the acoustic radiation pressure in the object. This enables deformation of tissue at the focus position to be detected directly and in real time. Alternatively, the light axis may be set at a position at a predetermined distance from the focus, and a basis for evaluation of viscoelasticity depending on the distance may be set.

The control computation unit may compute a predetermined mechanical feature quantity caused by deformation of tissue or a change in a mechanical feature quantity associated with deformation of tissue in association with the cross-sectional position in the object, and compute tomographic distribution of the viscoelasticity of tissue on the basis of the computation result. The “mechanical feature quantity” used herein may be obtained on the basis of a deformation quantity or a deformation vector of tissue. For example, the mechanical feature quantity may be a strain tensor obtained as a space derivative of a deformation vector. Alternatively, the mechanical feature quantity may be the amplitude or the phase of strain. Still alternatively, the mechanical feature quantity may be a deformation rate vector obtained as a time derivative of a deformation vector, or a strain rate tensor further obtained as a space derivative of the deformation rate vector. Still alternatively, the mechanical feature quantity may be the amplitude or the phase of any of these vectors or tensors. The viscoelasticity may be calculated on the basis of the amplitude of the exciting force caused by the acoustic radiation pressure, the amplitude of the strain detected by the OCT, and the phase difference therebetween. Because the exciting force caused by the acoustic radiation pressure is generated by driving of the loading device, the fact that the amplitude and the phase of the exciting force are known can be used.

The magnitude (amplitude value) of the variation of the deformation rate (strain rate) relative to the variation of the load and the following capability (responsiveness) of the variation of the deformation rate (strain rate) relative to the variation of the exciting force tend to change depending on the viscoelasticity of tissue. Specifically, as the viscosity is lower, the amplitude value of the deformation rate (strain rate) tends to be larger and the time lag (phase lag) tends to be smaller. The viscoelasticity can also be evaluated on the basis of these tendencies.

Alternatively, a shear wave caused by application of ultrasonic waves to the object may be tomographically detected, and the viscoelasticity of tissue may be computed from the propagation speed of the shear wave. The detection of the viscoelasticity of tissue is performed by the OCT using non-contact elastography using acoustic radiation pressure.

More specifically, the optical mechanism may emit light into the object through the front side thereof, and the loading device may output ultrasonic waves into the object through the rear side thereof. This facilitates prevention of physical interference between the optical mechanism and the loading device, and facilitates design of the devices.

For measuring regenerated tissue by such a configuration, a support for supporting a container containing the regenerated tissue may be provided. A translucent and transaudient container may be used as the container, and the support may have a hole through which the rear side of the container is exposed. This enables reception of light through the front side of the object and reception of ultrasonic waves through the rear side thereof. For quality assurance of regenerated tissue, the container is preferably sealable. The support may be located inside a clean bench.

The device may include a first optical mechanism, a second optical mechanism, and an optical detector. The first optical mechanism is provided on an object arm that includes the object, and guides light from a light source to the object to scan the object. The second optical mechanism is provided on a reference arm that does not include the object, and guides light from the light source to a reference mirror, so that the light is reflected by the reference mirror. The optical detector detects interference light resulting from superimposition of object light reflected by the object and reference light reflected by the reference mirror on each other. The control computation unit performs frequency analysis on an optical interference signal input from the optical detector, and calculates the deformation rate of tissue by using Doppler frequency. The control computation unit may then compute viscoelasticity on the basis of strain information obtained on the basis of the deformation rate and acoustic radiation pressure information from the loading device.

An example according to the present embodiment will now be described in detail with reference to the drawings.

### Example

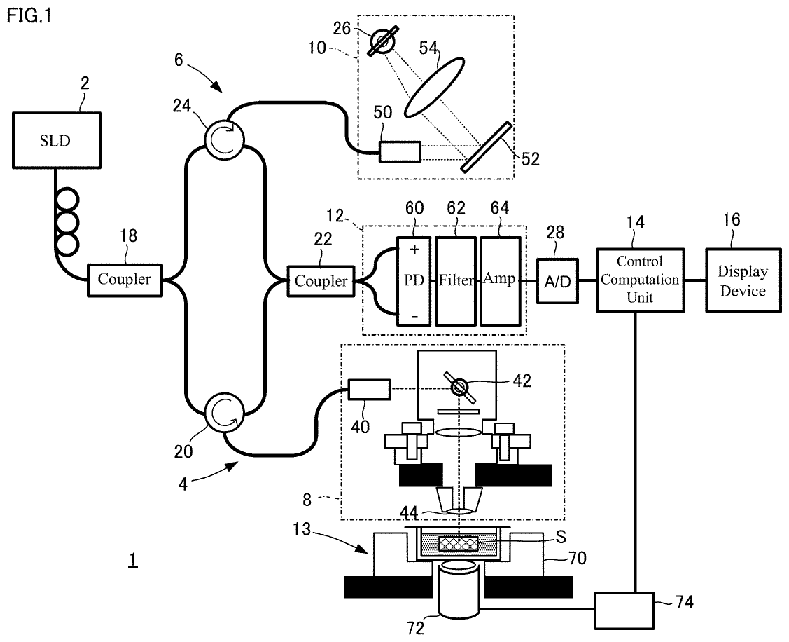

FIG. 1 is a diagram schematically illustrating a configuration of a viscoelasticity visualizing device according to the example. The device according to the example tomographically measures the viscoelasticity of regenerated tissue in microscale to visualize the viscoelasticity. For the tomographic measurement, load application by acoustic radiation pressure and detection by the OCT are used.

As illustrated in FIG. 1, an OCT device 1 includes a light source 2, an object arm 4, a reference arm 6, optical mechanisms 8 and 10, an optical detector 12, a loading device 13, a control computation unit 14, and a display device 16. The respective optical components are connected with one another by optical fibers. While an optical system based on a Mach-Zehnder interferometer is presented in the example illustrated in FIG. 1, other optical systems such as a Michelson interferometer may alternatively be used. In addition, while time domain OCT (TD-OCT) is used as the OCT in the present example, other types of OCT such as swept source OCT (SS-OCT) or spectral domain OCT (SD-OCT) may be used instead.

Light emitted from the light source 2 is split by a coupler 18 (beam splitter). One beam from the coupler 18 is guided to the object arm 4 and serves as object light, and the other is guided to the reference arm 6 and serves as reference light. The object light in the object arm 4 is guided to the optical mechanism 8 via a circulator 20, and directed to an object to be measured (hereinafter referred to as an “object S”). The object S is regenerated tissue (cultured tissue) in the present example. The object light is reflected as backscattered light at the surface and a cross section of the object S, returned to the circulator 20, and then guided to a coupler 22.

Meanwhile, the reference light at the reference arm 6 is guided to the optical mechanism 10 via a circulator 24. The reference light is reflected by a reference mirror 26 of the optical mechanism 10, returned to the circulator 24, and then guided to the coupler 22. Thus, the object light and the reference light are combined (superimposed) by the coupler 22, and interference light of the object light and the reference light is detected by the optical detector 12. The optical detector 12 detects the interference light as an optical interference signal (a signal indicating interference light intensity). The optical interference signal is input to the control computation unit 14 via an AD converter 28.

The control computation unit 14 performs control of the entire optical system, control of the loading device (ultrasonic vibration generator), and arithmetic processing for outputting images using the OCT. Command signals from the control computation unit 14 are input to the light source 2, the optical mechanisms 8 and 10, the loading device 13, etc. via a DA converter, which is not illustrated. The control computation unit 14 processes the optical interference signal input to the optical detector 12, and acquires a tomographic image of the object S using the OCT. The control computation unit 14 then computes tomographic distribution of the viscoelasticity of the object S on the basis of the tomographic image data by a technique described later.

The above will be described in more detail below.

The light source 2 is a broadband light source constituted by super luminescent diodes (hereinafter referred to as “SLDs”). Light emitted from the light source 2 is split by the coupler 18 into the object light and the reference light, which are guided to the object arm 4 and the reference arm 6, respectively. For the object arm 4 and the reference arm 6, polarization maintaining optical fibers capable of reducing the influence of polarization are used as optical fibers.

The optical mechanism 8 is included in the object arm 4, and includes a mechanism for guiding light from the light source 2 to the object S to scan the object S, and a drive unit for driving the mechanism. The optical mechanism 8 includes a collimator lens 40, a two-axis galvanometer mirror 42, and an object lens 44. The object lens 44 is arranged to face the object S. Light having passed through the circulator 20 is guided to the galvanometer mirror 42 via the collimator lens 40, scanned in the x-axis direction and the y-axis direction, and directed to the object S. Light reflected by the object S is returned as object light to the circulator 20, and guided to the coupler 22.

The optical mechanism 10 is a rapid scanning optical delay line (RSOD) mechanism included in the reference arm 6. The RSOD may have a single-pass configuration in which light is directed to a diffraction grating 52, which will be described later, once per one round trip thereof, or may have a double-pass configuration in which light is directed to the diffraction grating 52 twice per one round trip thereof. The optical mechanism 10 includes a collimator lens 50, the diffraction grating 52, a lens 54, and the reference mirror 26. Light having passed through the circulator 24 is guided to the diffraction grating 52 via the collimator lens 50. The light is split by the diffraction grating 52 according to wavelength, and focused on different positions on the reference mirror 26 by the lens 54. Rapid optical scanning and frequency modulation are possible by turning the reference mirror 26 by minute angles. Light reflected by the reference mirror 26 is returned as reference light to the circulator 24 and guided to the coupler 22. The light is then superimposed with the object light, and sent as interference light to the optical detector 12. In a modification, light coming via the diffraction grating 52 may be focused on the reference mirror 26 by a concave mirror instead of the lens 54.

The optical detector 12 includes a photodetector 60, a filter 62, and an amplifier 64. The interference light obtained through the coupler 22 is detected as an optical interference signal by the photodetector 60. The optical interference signal is subjected to denoising or noise reduction by the filter 62, and input to the control computation unit 14 via the amplifier 64 and the AD converter 28.

The loading device 13 includes a support 70 for holding the object S on the optical axis of the OCT, an ultrasound device 72 that outputs ultrasonic waves toward the object S, and a drive circuit 74 that drives the ultrasound device 72. As a result of the driving (oscillation) of the ultrasound device 72, an ultrasonic beam is output toward the inside of the object S. The drive circuit 74 causes the ultrasonic beam to scan vertically and horizontally in synchronization with the detection by the OCT on the basis of commands from the control computation unit 14. This enables a dynamic load (a vibration load, or exciting force) caused by the acoustic radiation pressure to be applied to any position in the tissue of the object S in a non-contact manner. The acoustic radiation pressure loading signal caused by the oscillation needs to be amplitude-modulated. Here, the amplitude that is directly dependent on the acoustic pressure amount (compressive load amount) of the acoustic radiation pressure is modulated by a sine wave, and the amplitude-modulated signal is transmitted to the object S. Because the amplitude modulation promotes deformation of tissue, the deformation rate of the tissue resulting from the modulation is detected by the OCT.

The control computation unit 14 includes a CPU, a ROM, a RAM, a hard disk, and the like. The control computation unit 14 performs, by the hardware and software, control of the entire optical system, drive control of the loading device 13, and arithmetic processing for outputting images by the OCT. The control computation unit 14 controls driving of the loading device 13 and the optical mechanisms 8 and 10, processes the optical interference signal output from the optical detector 12 on the basis of the driving, and acquires tomographic images of the object S obtained by the OCT. The control computation unit 14 then computes the viscoelasticity of the tissue of the object S by a technique described below on the basis of the tomographic image data.

The display device 16 is constituted by a liquid crystal display, for example, and displays, on a screen, the viscoelasticity of the tissue of the object S computed by the control computation unit 14 in a form of tomographic visualization.

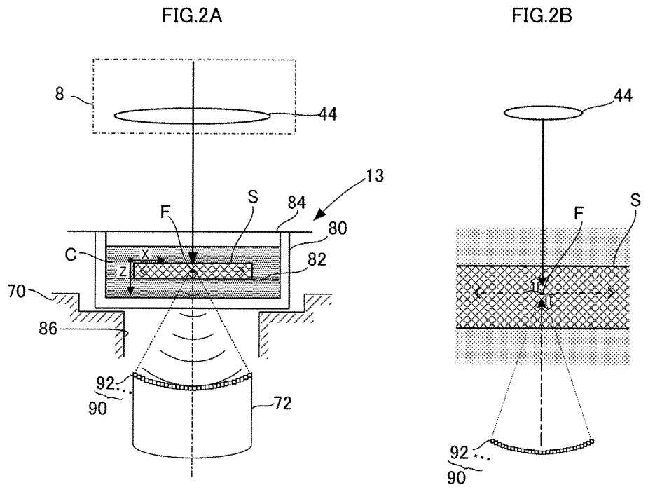

FIGS. 2A and 2B are explanatory diagrams illustrating a method for applying load to the object S in a non-contact manner. FIG. 2A schematically illustrates the loading device 13 and components therearound. FIG. 2B illustrates a state in which acoustic radiation pressure is loaded.

As illustrated in FIG. 2A, the object S is accommodated in a container 80, and the container 80 is placed on the support 70. This is to prevent contamination while load application and OCT measurement because the object S is regenerated tissue in the present embodiment. The container 80 accommodates the object S in a state in which a substrate 82 on which the object S is disseminated is immersed in a culture medium C. The container 80 is closed (sealed) by a cover 84. The container 80, the substrate 82, and the cover 84 are made of translucent and transaudient materials. Note that at least part of the optical mechanism 8 and the loading device 13 is preferably accommodated in the clean bench, so that contamination of the inside of the container 80 by dust or unwanted bacteria is reliably prevented.

The support 70 has a through-hole 86 in the vertical direction. The container 80 is placed on the support 70 across the through-hole 86, through which the rear side of the container 80 is exposed. The object S is positioned so that at least a measurement range thereof is included in a plane of projection of the through-hole 86 in the axial direction. The ultrasound device 72 faces the rear face of the container 80 with the through-hole 86 therebetween, and outputs ultrasonic waves toward the object S.

The ultrasound device 72 includes a curved transducer array 90 on a top face thereof. The transducer array 90 includes a plurality of elements 92 arranged in a curved face. The elements 92 are constituted by ultrasonic elements (piezoelectric elements) that generate ultrasonic waves for excitation. As also illustrated in FIG. 2B, the outputs of ultrasonic waves from the elements 92 are adjusted, so that the acoustic radiation pressure is focused on a given position, which is referred to as a focus point F, in the object S and excites (displaces) the tissue at the focus point F (see white arrows). The generation of the acoustic radiation pressure causes a shear wave starting from the focus point F and in a direction parallel to the surface of the object S (see a two-dot chain arrow). In the present example, the axis of light produced by the OCT is set to pass through the focus point F of the object S. The position of the focus point F can be adjusted by selecting elements 92 to be driven from among the elements 92. In other words, the position of the focus point F can be scanned by changing elements 92 to be energized from among the elements 92. Note that, in a modification, the ultrasonic elements may be arranged in a planar shape or the like, and the acoustic radiation pressure may be focused on the focus point F by an acoustic lens.

The control computation unit 14 sets the focus point F in the object S on which the acoustic radiation pressure is to be focused, the ultrasonic elements to be driven, excitation frequency caused by the acoustic radiation pressure, and the like, and outputs control signals (pulses) to the drive circuit 74 on the basis of the settings. As a result, drive signals (pulses) are output from the drive circuit 74 to the ultrasound device 72, and ultrasonic waves (excitation pulses) are output toward the focus point F.

A method for arithmetic processing of the viscoelasticity of the object S will now be explained in detail below.

As described above, according to the OCT, the object light (reflected light from the object) having passed through the object arm 4 and the reference light having passed through the reference arm 6 are combined, and detected as an optical interference signal by the optical detector 12. The control computation unit 14 is capable of acquiring the optical interference signal as a tomographic image of the object S based on the interference light intensity. While the tomographic distribution can be computed three-dimensionally, one-dimensional or two-dimensional computation thereof will be explained here.

A coherence length 1, which represents the resolution in the optical axis direction (depth direction) of the OCT is determined by an autocorrelation function of the light source. Herein, the coherence length l_{c} is the half width at half maximum of the envelope of the autocorrelation function, and can be expressed by the following expression (1).

$${l}_{c} = \frac{2 ln 2}{π} ( \frac{{λ}_{c}^{2}}{Δ λ} ) \qquad ( 1 )$$

In the expression, λ_{c} represents the center wavelength of a beam, and Δλ represents the full width at half maximum of the beam.

In addition, the resolution in the direction perpendicular to the optical axis (beam scanning direction) is ½ of a beam-spot diameter D on the basis of the focusing performance of a focusing lens. The beam-spot diameter ΔΩ can be expressed by the following expression (2).

$$Δ Ω = \frac{4 {λ}_{c}}{π} ( \frac{f}{d} ) \qquad ( 2 )$$

In the expression (2), d represents the diameter of a beam incident on the focusing lens, and f represents the focal length of the focusing lens.

A Doppler modulated signal is detected by the OCT, which enables calculation of deformation rate distribution of tissue. Specifically, the electric field E′ᵣ(t) of the reference light is expressed by the following expression (3).

Eᵣ′(t)=A(t)exp{i(−(w_{c}+wᵣ)t)} (3)

In the expression (3), A(t) represents amplitude, and ω_{c} represents the central angular frequency of the light source. ωᵣ represents a Doppler angular frequency generated by turning of the reference mirror 26 of the RSOD.

In view of a Doppler angular frequency shift amount ω_{d} caused depending on the deformation rate of tissue, the electric field E′ₒ(t) of the object light is expressed by the following expression (4).

E′ₒ(t)=∫A(t−τ)R(z)exp{i(−(ω_{c}+ω_{d})(t−τ))}d_{τ} (4)

Because light intensity is the time average of the square of electric field intensity, a detected interference light intensity I′_{d}(t) is expressed by the following expression (5).

$$\begin{matrix}{I}_{d}^{′} ( t ) = {〈 {\vert {E}_{r}^{′} ( t ) + {E}_{o}^{′} ( t ) \vert}^{2} 〉}_{τ} \\ = {〈 {\vert {E}_{r}^{′} ( t ) \vert}^{2} 〉}_{τ} + {〈 {\vert {E}_{o}^{′} ( t ) \vert}^{2} 〉}_{τ} + 2 {〈 \vert {{E}_{r}^{′} ( t )}^{*} {E}_{o}^{′} ( t ) \vert 〉}_{τ} \\ = {I}_{r}^{′} + {I}_{o}^{′} \\ + 2 Re ( \int R ( z ) {〈 A ( t ) A ( t - τ ) 〉}_{τ} exp \{ i ( {ω}_{c} + {ω}_{d} ) τ \} exp \{ i ( {ω}_{r} - {ω}_{d} ) t \} dr )\end{matrix} \qquad ( 5 )$$

In addition, a detected tomographic interference signal I_{d}(x,y,z) is expressed by the following expression (6).

$$\begin{matrix}{I}_{d} ( x , y , z ) = R ( z ) ⊗ G ( z ) exp ( i \frac{2 π}{{λ}_{c}} z ) exp ( i ( {ω}_{r} - {ω}_{d} ) t ) \\ = R ( z ) ⊗ ( {I}_{m}^{′} exp \{ - \frac{\sqrt{ln 2}}{{Δz}^{2}} {z}^{2} \} ) exp ( i ( {ω}_{r} - {ω}_{d} ) t )\end{matrix} \qquad ( 6 )$$

The angular frequency of an interference signal is expressed as ωᵣ−ω_{d}. The carrier angular frequency ωᵣ of the interference signal is used to detect the Doppler angular frequency shift amount ω_{d} generated depending on the deformation rate. The following expression (7) can then be computed by using the detected Doppler angular frequency shift amount ω_{d} to obtain the deformation rate v.

$$v = \frac{{ω}_{d} {λ}_{c}}{4 π n cos θ ( x , y , z )} \qquad ( 7 )$$

In the expression (7), λ_{c} represents the center wavelength of the light source, θ(x,y,z) represents an angle between the deformation rate direction and the beam incidence direction at coordinates (x,y,z). n represents the average refractive index in the tissue.

[Adjacent Autocorrelation Method]

In the present example, the technique of scanning in the depth direction (z axis direction) using the RSOD is employed as described above. In order to prevent degradation in deformation rate detection performance in this process, the Hilbert transform and the adjacent autocorrelation method are applied. Specifically, the adjacent autocorrelation method is applied to analytic signals (complex signals) Γ(t) obtained by applying the Hilbert transform to spatially adjacent interference signals. In this manner, a phase difference Δφ at certain coordinates at predetermined time intervals (time difference ΔT) is obtained, and a Doppler angular frequency shift amount ω_{d} depending on the deformation rate is detected.

Specifically, the respective interference signals are expressed as the following expression (8) where j-th and (j+1)-th analytic signals obtained by the application of the Hilbert transform are represented by Γⱼ and Γⱼ₊₁, respectively.

$$\begin{matrix}{Γ}_{j} ( t ) = {s}_{j} ( t ) + i {\overline{s}}_{j} ( t ) \\ = A ( t ) exp \{ i ( {ω}_{r} ( λ ) ) t + {ω}_{d} t \} \\ {Γ}_{j + 1} ( t + Δ T ) = {s}_{j + 1} ( t + Δ T ) + i {\hat{s}}_{j + 1} ( t + Δ T ) \\ = A ( t ) exp \{ i ( {ω}_{r} ( λ ) ) t + {ω}_{d} ( t + Δ T ) \}\end{matrix} \qquad ( 8 )$$

In the expression (8), s(t) represents the real part of the analytic signal Γ(t), and s\hat{ }(t) represents the imaginary part thereof. AT represents a time interval between acquisitions of the j-th and the (j+1)-th interference signals, and A represents the amplitude (that is, backscatter intensity) of the interference signals.

In a case where phase modulation has not occurred in the electric field of the object light (ω_{d}=0), the phase difference of the interference signals is zero. The phase difference between the interference signals of Γⱼ and Γⱼ₊₁ corresponds to the phase shift amount ω_{d}ΔT resulting from Doppler modulation caused depending on the deformation rate. Specifically, the Doppler angular frequency shift amount ω_{d} caused depending on the deformation rate is expressed as the following expression (9).

$$\begin{matrix}{ω}_{d} = \frac{1}{Δ T} \cdot {ω}_{d} \cdot Δ T \\ = \frac{1}{Δ T} \cdot Δ φ \\ = \frac{1}{Δ T} \cdot ( {φ}_{j + 1} - {φ}_{j} ) \\ = \frac{1}{Δ T} \cdot arctan ( {Γ}_{j + 1} {Γ}_{j}^{*} ) \\ = \frac{1}{Δ T} \cdot arctan ( \frac{{s}_{j + 1} {\hat{s}}_{j} - {s}_{j} {\hat{s}}_{j + 1}}{{s}_{j + 1} {s}_{j} + {\hat{s}}_{j + 1} {\hat{s}}_{j}} )\end{matrix} \qquad ( 9 )$$

Since the angle of deviation is Δφ=ω_{d}ΔT, the phase shift amount can be detected in a range of −π to π. In addition, ensemble average can be performed on n interference signals according to the following expression (10) to improve the detection performance of the Doppler angular frequency shift amount ω_{d}.

$${ω}_{d} = \frac{1}{Δ T} arctan ( \frac{Im ( {\sum }_{j = 1}^{n} {Γ}_{j + 1} {Γ}_{j}^{*} )}{Re ( {\sum }_{j = 1}^{n} {Γ}_{j + 1} {Γ}_{j}^{*} )} ) \qquad ( 10 )$$

The Doppler angular frequency shift amount ω_{d} obtained as described above is substituted into the expression (7) to calculate the deformation rate v. The viscoelasticity of the tissue can be computed on the basis of the deformation rate v.

Specifically, the complex elastic modulus of a viscoelastic material is known to be expressed by E* in expression (11) below. The real part E′ of the complex elastic modulus E* corresponds to a component with stress and strain being in the same phase, and the imaginary part E″ thereof corresponds to a component with phases of stress and strain being shifted by 90 degrees from each other. The former E′ corresponds to an elastic component and is called a “storage elastic modulus” (hereinafter referred to as a “storage elastic modulus E′”). The latter E″ corresponds to a viscous component with stress and strain rate being in the same phase and is called a “loss elastic modulus” (hereinafter referred to as a “loss elastic modulus E″”). The distribution of the storage elastic modulus E′ and the loss elastic modulus E″ can thus be computed as distribution of viscoelasticity.

Note that the strain amount (strain rate) at each clock time can be calculated from the distribution of the deformation rate v of the tissue (distribution in the Z direction) by the least-squares method, and the amplitude (strain amplitude εₒ) of the strain amount can be tomographically detected. In addition, the magnitude (stress amplitude σ_{d} of the acoustic radiation pressure of the ultrasonic waves is a controlled variable set for the ultrasound device 72 by the control computation unit 14, and can thus be obtained. Because the oscillation timing of ultrasonic waves and the OCT measurement timing are known by the control computation unit 14, the phase difference (loss angle δ) between stress and strain can be calculated. Each of the storage elastic modulus E′ and the loss elastic modulus E″, which are viscoelasticity parameters, can thus be calculated by the following expression (11).

E*=(σ₀/ε₀)e^{iδ}=E′+iE″

E′=|E*|cos δ

E″=|E*|sin δ (11)

The tomographic distribution of the thus obtained storage elastic modulus E′ and loss elastic modulus E″ virtually presents the tomographic distribution of the viscoelasticity of the tissue. The control computation unit tomographically visualizes the viscoelasticity of the tissue.

In addition to the processing described above, the viscoelasticity of the tissue can be tomographically visualized in a two-dimensional or three-dimensional manner, which is post-processing after acquiring two-dimensional tomographic images in advance. This is a technique of applying a digital cross-correlation method to two OCT images acquired before and after deformation of the object to calculate distribution of deformation vector, and tomographically detecting distribution of strain rate tensor of the tissue in microscale.

For calculation of the deformation vector distribution, a recursive cross-correlation method of repeating cross-correlation is applied. This is a technique of applying a cross-correlation method by referring to deformation vectors calculated at low resolution, and limiting a search region while hierarchically reducing an interrogation region (subset, inspection region). This enables high-resolution deformation vectors to be obtained. Furthermore, an adjacent cross-correlation multiplication of multiplying correlation value distributions of adjacent interrogation regions is used as a method for reducing speckle noise. Correlation value distribution with a higher SNR obtained by the multiplication is then searched for a maximum correlation value.

In microscale deformation analysis, sub-pixel accuracy of deformation vectors is important. Thus, sub-pixel analysis methods including an upstream gradient method using an intensity gradient and an image deformation method based on expansion/contraction and shear are used to achieve high accuracy detection of deformation vectors. Note that the “upstream gradient method” used herein is one type of gradient methods (optical flow methods).

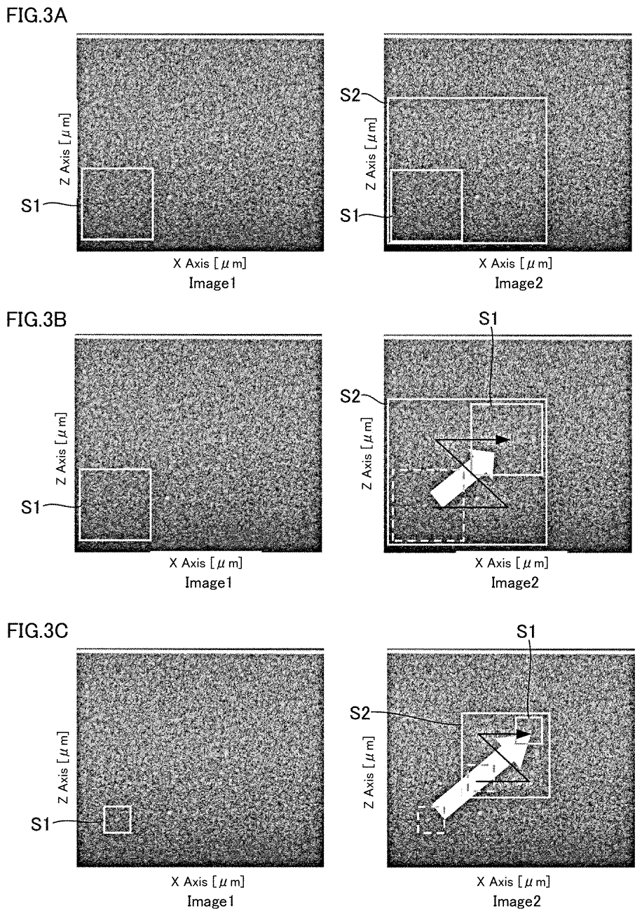

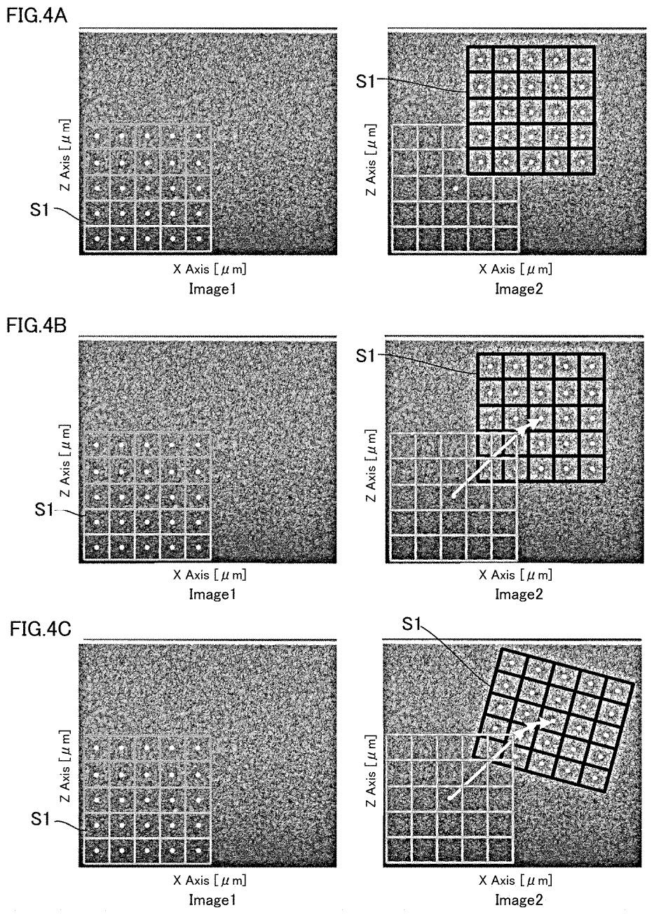

FIGS. 3A to 3C schematically show procedures of the recursive cross-correlation method. FIGS. 4A to 4C schematically show procedures of sub-pixel analysis.

[Recursive Cross-Correlation Method]

FIGS. 3A to 3C show processes of the recursive cross-correlation method. Each of FIGS. 3A to 3C shows tomographic images taken continuously using the OCT. A left image shows a tomographic image (Image1) taken first, and a right image shows a tomographic image (Image2) taken next.

A cross-correlation method is a method of evaluating similarity of local speckle patterns by using a correlation value Rᵢⱼ according to expression (12) below. Thus, as shown in FIG. 3A, in the OCT images taken continuously, an interrogation region S1 for similarity inspection is set in the first tomographic image (Image1), and a search region S2, which is a range for searching similarity, is set in the next tomographic image (Image2).

$$\begin{matrix}{R}_{i , j} ( Δ X , Δ Z ) = {\sum }_{i = 1}^{{N}_{x}} {\sum }_{j = 1}^{{N}_{z}} \{ f ( {X}_{i} , {Z}_{j} ) - \overline{f} \} \{ g ( {X}_{i} + Δ X , {Z}_{j} + Δ Z ) - \ddot{g} \} \\ \times {( {\sum }_{i = 1}^{{N}_{x}} {\sum }_{j = 1}^{{N}_{z}} {\{ f ( {X}_{i} , {Z}_{j} ) - \ddot{f} \}}^{2} )}^{- \frac{1}{2}} \\ \times {( {\sum }_{i = 1}^{{N}_{x}} {\sum }_{j = 1}^{{N}_{z}} {( g ( {X}_{i} + Δ X , {Z}_{j} + Δ Z ) - \dot{g} \}}^{2} )}^{- \frac{1}{2}}\end{matrix} \qquad ( 12 )$$

Note that a spatial coordinate system is set, in which a Z axis is the optical axis direction, and an X axis is perpendicular to the Z axis. f(Xᵢ,Zⱼ) and g(Xᵢ,Zⱼ) represent speckle patterns in the interrogation region S1 (Nₓ×N_{z} pixels) at a center position (Xᵢ,Zⱼ) of the OCT images before and after deformation.

Note that f⁻ and g⁻ represent average values of f(Xᵢ,Zⱼ) and g(Xᵢ,Zⱼ) within the interrogation region S1.

Correlation value distribution R_{i,j}(ΔX,ΔZ) in the search region S2 (Mₓ×M_{z} pixels) is calculated, and pattern matching is carried out as shown in FIG. 3B. In practice, a displacement U_{i,j} that produces the maximum correlation value is determined to be a deformation vector between before and after deformation as expressed by the following expression (13).

$${{U}^{->}}_{ij} = ( \begin{matrix}Δ {X}_{ma x} \\ Δ {Z}_{ma x}\end{matrix} ) = arg max {R}_{i , j} ( Δ X , Δ Z ) \qquad ( 13 )$$

This technique employs the recursive cross-correlation method of repeating a cross-correlation process while reducing the interrogation region S1 to increase spatial resolution. Note that, in the present example, the resolution is increased such that the spatial resolution is doubled. As shown in FIG. 3C, the interrogation region S1 is divided to be reduced to ¼, the deformation vector calculated in the previous hierarchy is referred to, and the search region S2 is reduced. The search region S2 is also divided to be reduced to ¼. Use of the recursive cross-correlation method prevents or reduces erroneous vectors that are frequently generated at high resolution. Such a recursive cross-correlation process increases the resolution of deformation vectors.

In addition, a threshold using a mean deviation σ of a total of nine deformation vectors including the coordinate being calculated and eight coordinates around the calculated coordinate is set so as to remove erroneous vectors and prevent error propagation due to the recursive processes by the following expression (14).

|\vec{U}_{i,j}−\vec{U}ₘ|≤κσ (14)

In the expression (14), Uₘ represents a median of vector quantity, and a coefficient K that is the threshold is freely set.

[Adjacent Cross-Correlation Multiplication]

In the present example, the adjacent cross-correlation multiplication is introduced as a method for determining an accurate maximum correlation value from highly-random correlation value distribution affected by speckle noise. The adjacent cross-correlation multiplication multiplies the correlation value distribution R_{i,j}(Δx,Δz) in the interrogation region S1 by R_{i+Δi,j}(Δx,Δz) and R_{i,j+Δj}(Δx,Δz) for adjacent interrogation regions overlapping with the interrogation region S1 to search for a maximum correlation value by using a new correlation value distribution R′_{i,j}(Δx,Δz), as expressed by the following expression (15).

$$\begin{matrix}{R}_{i , j}^{′} ( Δ x , Δ z ) = {R}_{i , j} ( Δ x , Δ z ) \\ \times {R}_{i + Δ ij} ( Δ x , Δ z ) \times {R}_{i , j + Δj} ( Δ x , Δ z )\end{matrix} \qquad ( 15 )$$

As a result, the multiplication of the correlation values enables reduction in the randomness. Since the amount of information of interference intensity distribution is also reduced with the aforementioned reduction in size of the interrogation region S1, occurrence of multiple correlation peaks caused by speckle noise is considered to result in degradation in measurement accuracy. In contrast, since the displacements of adjacent boundaries are correlated, a high correlation value remains near the coordinates of the maximum correlation value. The introduction of the adjacent cross-correlation multiplication makes the maximum correlation peak clearer, improves the measurement accuracy, and accurately extracts displaced coordinates. In addition, introduction of the adjacent cross-correlation multiplication at respective stages of the OCT prevents or reduces error propagation, and improves robustness to speckle noise. As a result, accurate deformation vectors and strain distribution can be calculated even at high spatial resolution.

[Upstream Gradient Method]

FIGS. 4A to 4C show processes according to sub-pixel analysis. Each of FIGS. 4A to 4C shows tomographic images taken continuously by the OCT. A left image shows a tomographic image (Image 1) taken first, and a right image shows a tomographic image (Image 2) taken next.

In the present example, an upstream gradient method and an image deformation method are used for the sub-pixel analysis. Although a final displacement is calculated by the image deformation method to be described below, the upstream gradient method is applied prior to the image deformation method for the reason of convergence of calculations. The image deformation method and the upstream gradient method for detecting a sub-pixel displacement with high accuracy are applied under a condition where the interrogation region size is small and the spatial resolution is high. In a case where detection of a sub-pixel displacement by the image deformation method is difficult, the sub-pixel displacement is calculated by the upstream gradient method.

In the sub-pixel analysis, an intensity difference between before and after deformation at a point of interest is expressed by the intensity gradients of respective components and the displacement. Thus, the sub-pixel displacement can be determined by using the least-squares method from intensity gradient data in the interrogation region S1. In the present example, for obtaining an intensity gradient, an upwind difference method that provides an upwind intensity gradient before sub-pixel deformation is used.

The sub-pixel analysis is highly dependent on speckle noise that is not only caused by measurement errors but intricately related to the scattering effect and the polarization characteristics of the tissue. Furthermore, because a spatial derivative of the displacement is needed for calculation of the strain tensor distribution, sub-pixel errors increase the numerical instability of derivative calculation, and make the strain tensor distribution unstable. While there are various techniques for sub-pixel analysis, the gradient method that detects the sub-pixel displacement with high accuracy even under the condition where the interrogation region size is small and the spatial resolution is high is used in the present example.

The upstream gradient method is a technique for calculating displacement of a point of interest within the interrogation region S1 not just with pixel accuracy as shown in FIG. 4A but with sub-pixel accuracy as shown in FIG. 4B. Note that each square in FIGS. 4A to 4C represents one pixel. Although the pixels are actually significantly smaller than the shown tomographic images, the pixels are illustrated in a large size for convenience of explanation. The upstream gradient method is a technique of formulating a change in intensity distribution between before and after small deformation by using the intensity gradient and the displacement, and is expressed by the following expression (16) that calculates the Taylor expansion of small deformation f(x+Δx,z+Δz) where f represents the intensity.

fₓ(x,z)Δx+f_{z}(x,z)Δz=f(x+Δx,z+Δz)−f(x,z) (16)

The expression (16) shows that the intensity difference at the point of interest between before and after deformation is expressed by the intensity gradient before the deformation and the displacement. Note that, because the displacement (Δx,Δz) cannot be determined only by the expression (16), the displacement within the interrogation region S1 is assumed to be constant and the least-squares method is applied for the calculation.

In calculation of the displacement by using the expression (16), the intensity difference at each point of interest between before and after displacement on the right side of the expression (16) is obtained only uniquely. Thus, the accuracy of intensity gradient calculation is directly linked to the accuracy of the displacement. For differencing of the intensity gradient, first-order upwind difference is used. This is because a large amount of data will be required and the influence of contained noise, if any, will be great if a high-order difference is applied to differencing. This is also because a large amount of data outside of the interrogation region S1 will be used in a high-order difference based on a point in the interrogation region S1, which poses a problem that the obtained displacement will not be an amount of the interrogation region S1 itself.

In obtaining the intensity gradient, because it can be assumed that the displacement of an upwind intensity gradient before deformation causes an intensity difference at a point of interest, an upwind difference is applied before deformation. The term upwind used herein does not mean the actual displacement direction but means the direction of a sub-pixel displacement with respect to a pixel displacement, and the upwind direction is determined by applying parabolic approximation to the maximum correlation peak. Conversely, because it can be assumed that the reverse displacement of a downwind intensity gradient after deformation causes an intensity difference at a point of interest, a downwind difference is applied after deformation.

Two solutions are found by using the upwind difference before deformation and the downwind difference after deformation, and an average of the solutions is obtained. Furthermore, the intensity gradients before and after deformation are not actually on the same axis as the points of interest if the displacements are not along the axial direction, and gradients at shifted positions need to be obtained. Specifically, for obtaining a gradient in the X direction, displacement in the Z direction need to be taken into consideration. Thus, the intensity gradient is estimated by intensity interpolation, so as to improve the accuracy. Basically, a position before (or after) deformation is estimated, and the gradient at the position is obtained by interpolation.

The position of a point of interest before (or after) deformation is obtained from the sub-pixel displacement when the parabolic approximation is applied. Eight coordinates surrounding the point of interest position are used, and the intensity gradient is calculated from the ratios of the intensities at the eight coordinates to the intensity at the point of interest. Specifically, the following expression (17) is used. The least-squares method is applied using the thus calculated intensity gradient and an intensity change to determine the displacement.

$$\begin{matrix}{f}_{x} ( x , z ) = Δ z \{ f ( x , z - 1 ) - f ( x - 1 , z - 1 ) \} \\ + ( 1 - Δ z ) \{ f ( x , z ) - f ( x - 1 , z ) \}\end{matrix} \qquad ( 17 )$$

[Image Deformation Method]

In the calculation of deformation vectors until the upstream gradient method described above, the shape of the interrogation region S1 has not been changed and remains square. In reality, however, because the interrogation region S1 is likely to be deformed with the deformation of the object, an algorithm based on small deformation of the interrogation region S1 needs to be introduced for calculation of deformation vectors with high accuracy. Thus, in the present example, the image deformation method is introduced for calculation of deformation vectors with sub-pixel accuracy. Specifically, cross-correlation between the interrogation region S1 before tissue deformation and the interrogation region S1 after the tissue deformation where expansion/contraction and shear deformation is considered is performed, and a sub-pixel deformation quantity is determined by repeated calculation based on the correlation value. Note that the expansion/contraction and shear deformation of the interrogation region S1 is linearly approximated.

The image deformation method is typically used for surface strain measurement of a material, and applied to an image of a material surface coated with a random pattern taken by a high spatial resolution camera. In contrast, an OCT image not only contains much speckle noise, but also deforms greatly with respect to speckle patterns because matrices and moisture in tissue flow and the refractive index thus changes particularly in biological tissue. Thus, the convergence of the solution is significantly lowered if local deformation occurs in complex tissue and is accompanied by deformation such as expansion, contraction, and shear in the interrogation region S1. The reduction of the interrogation region S1 in the present technique is essential for detection of a local, tissue mechanical characteristic. Thus, in the image deformation method, the deformation quantity obtained by the upstream gradient method is used as an initial value for convergence calculation, and bicubic interpolation of the intensity distribution is further used, to achieve low robustness even when the interrogation region S1 is reduced. Alternatively, in a modification, an interpolation function other than the bicubic interpolation may be used.

More specifically, the computation is performed according to the following procedures. First, the bicubic interpolation is applied to the intensity distribution of the OCT image before the tissue deformation, to make the intensity distribution continuous. The bicubic interpolation is a technique of using a convolution function obtained by piecewise approximation of cubic polynomial function of a sinc function, to reproduce spatial continuity of intensity information. Because a point spread function dependent on an optical system is convolved in image measurement of intensity distribution, which is originally continuous, the originally continuous intensity distribution is reproduced by deconvolution using the sinc function. For interpolation of a single-axis, discrete signal f(x), a convolution function h(x) is expressed by the following expression (18).

$$h ( x ) = \{ \begin{matrix}( a + 2 ) {\vert x \vert}^{3} - ( a + 3 ) {\vert x \vert}^{2} + 1 & ( \vert x \vert \leq 1 ) \\ a {\vert x \vert}^{3} - 5 a {\vert x \vert}^{2} + 8 a \vert x \vert - 4 a & ( 1 \leq \vert x \vert \leq 2 ) \\ 0 & ( \vert x \vert \geq 2 )\end{matrix} \qquad ( 18 )$$

Note that the shape of the interpolation function h(x) also needs to be changed depending on the difference in OCT measurement condition. An algorithm in which a derivative a of the intensity interpolation function h(x) where x=1 is made to be variable and in which the shape of the intensity interpolation function h(x) is changeable by changing the value a is thus provided. In the present example, the value a is determined on the basis of a result of verification by numerical experiment using a pseudo-OCT image. The image interpolation as described above allows an OCT intensity value to be obtained at each point in the interrogation region S1 where expansion/contraction and shear deformation is considered.

The interrogation region S1 calculated in view of expansion/contraction and shear deformation is deformed with displacement as shown in FIG. 4C. When it is assumed that coordinates (x,z) at an integer pixel position in an interrogation region S1 of an OCT image before tissue deformation are displaced to coordinates (x*,z*) after tissue deformation, the values of x* and z* are expressed by the following expression (19).

$$\{ \begin{matrix}{x}^{*} = x + Δ x + u + \frac{\partial u}{\partial x} Δ x + \frac{\partial u}{\partial z} Δ z \\ {z}^{*} = z + Δ z + v + \frac{\partial v}{\partial x} Δ x + \frac{\partial v}{\partial z} Δx + \frac{\partial v}{\partial z} Δz\end{matrix} \qquad ( 19 )$$

In the expression (19), Δx and Δz represent the distances from the center of the interrogation region S1 to coordinates x and z, respectively, u and v represent deformation quantities in the x and z directions, respectively, ∂u/∂x and ∂v/∂z represent vertical strains in the x and y directions of the interrogation region S1, respectively, and ∂u/∂z and ∂v/∂x represent shear strains in the x and y directions of the interrogation region S1, respectively. The Newton-Raphson method is used for numerical solution, and repeated calculation is carried out so that the correlation value derivative with six variables (u, v, au/ax, ∂u/∂z, ∂v/∂x, and ∂v/∂z) becomes 0, that is, so that the maximum correlation value is obtained. In order to increase the convergence of the repeated calculation, sub-pixel displacements obtained by the upstream gradient method are used for displacement initial values in the x and z directions. When a Hessian matrix for a correlation value R is represented by H, and a Jacobi vector for the correlation value is represented by ∇R, an update amount ΔPi obtained at each repetition is expressed by the following expression (20).

ΔPᵢ=−H⁻¹∇R (20)

The determination of convergence is based on the fact that the asymptotic solution obtained at each repetition of calculation becomes sufficiently small near the convergent solution. In a region where the speckle pattern changes drastically, however, a correct convergent solution may not be obtained because the change cannot be tracked by linear deformation. In this case, the sub-pixel displacement obtained by the upstream gradient method is used in the present example. The deformation vectors with sub-pixel accuracy obtained as described above are differentiated with respect to time, which enables calculation of distribution of deformation rate vector.

[Temporal/Spatial Moving Least-Squares Method]

For calculation of a strain rate tensor, the moving least-squares method (hereinafter referred to as the “MLSM”) is used. The MLSM is a technique allowing smoothing of displacement distribution and stable calculation of a derivative. A squared error equation used in the MLSM is expressed by the following expression (21).

$$S ( x , z , t ) = {\sum }_{n = 1}^{N} {\sum }_{m = 1}^{M} {\sum }_{l - 1}^{L} {[ y ( {x}_{n} , {z}_{m} , {t}_{l} ) - f ( {x}_{n} , {z}_{m} \cdot {t}_{l} ) ]}^{2} \qquad ( 21 )$$

In the expression (21), parameters a to k with which S(x,z,t) is minimized are obtained. Specifically, the expression (22) below is used as a quadratic polynomial with three variables in the horizontal direction x, the depth direction z and the time direction t for the approximation function. An optimum derivative is then calculated by the following expression (23) on the basis of the least squares approximation, and smoothing is performed.

$$\begin{matrix}f ( {x}_{n} , {z}_{m} , {t}_{l} ) = a {x}_{n}^{2} + {bz}_{m}^{2} + {ct}_{l}^{2} + {dx}_{n} {z}_{m} + {ex}_{n} {t}_{l} + {gz}_{m} {t}_{l} + {hx}_{n} + {iz}_{m} + {jt}_{l} + k & ( 22 ) \\ ( \begin{matrix}\frac{\partial S ( x , y , z )}{\partial a} \\ ⋮ \\ \frac{\partial S ( x , y , z )}{\partial k}\end{matrix} ) + ( \begin{matrix}\frac{{\partial }^{2} S ( x , y , z )}{\partial {a}^{2}} & … & \frac{{\partial }^{2} S ( x , y , z )}{\partial a \partial k} \\ ⋮ & ⋱ & ⋮ \\ \frac{{\partial }^{2} S ( x , y , z )}{\partial k \partial a} & … & \frac{{\partial }^{2} S ( x , y , z )}{\partial {k}^{2}}\end{matrix} ) ( \begin{matrix}a \\ ⋮ \\ k\end{matrix} ) = ( \begin{matrix}0 \\ ⋮ \\ 0\end{matrix} ) & ( 23 )\end{matrix}$$

The strain rate tensor expressed by the following expression (24) can be calculated by using this derivative. fₓ and f_{z} represent the amounts by which strain is increased along the respective axes. The strain rate is calculated from the amounts of strain changes with respect to time.

$$\frac{\partial ϵ}{\partial t} = \frac{\partial }{\partial t} ( \begin{matrix}\frac{\partial {f}_{x}}{\partial x} & \frac{\partial {f}_{z}}{\partial x} \\ \frac{\partial {f}_{x}}{\partial z} & \frac{\partial {f}_{z}}{\partial z}\end{matrix} ) \qquad ( 24 )$$

The strain amount can be calculated by time integration of the strain rate calculated by the expression (24) above, and the amplitude (strain amplitude ε0) of the strain amount can be tomographically detected. In addition, as described in relation to the expression (11) above, the amplitude (stress amplitude σ0) of the acoustic radiation pressure of the ultrasonic waves can be given as a modulation amplitude by the control computation unit 14 or the drive circuit 74, and is therefore known. The phase difference (loss angle δ) between stress and strain can also be calculated. Thus, the storage elastic modulus E′ and the loss elastic modulus E″, which are viscoelasticity parameters, can be calculated. The tomographic distribution of the thus obtained storage elastic modulus E′ and loss elastic modulus E″ virtually presents the tomographic distribution of the viscoelasticity of tissue. The control computation unit 14 tomographically visualizes the viscoelasticity of the tissue. Note that formulation is also possible using the strain rate. In other words, the association between the strain rate and the viscoelasticity parameters can be obtained.

Next, a specific processing flow performed by the control computation unit 14 will be explained.

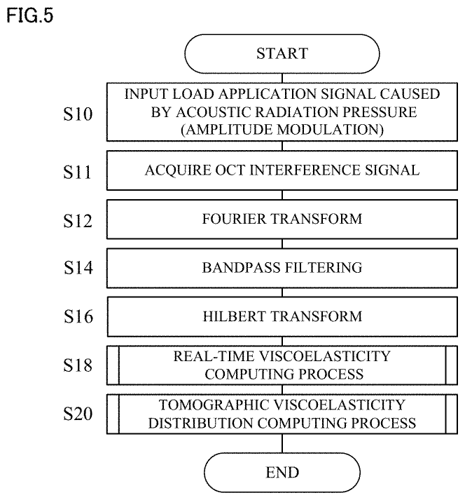

FIG. 5 is a flowchart illustrating a flow of a process of tomographically visualizing viscoelasticity performed by the control computation unit 14. This process is repeated every predetermined computation period. The control computation unit 14 controls driving of the light source 2, the optical mechanisms 8 and 10, and the loading device 13. The control computation unit 14 thus inputs an acoustic radiation pressure loading signal that is properly subjected to amplitude modulation (S10), and acquires an optical interference signal (S11) by the OCT.

The control computation unit 14 performs Fourier transform on the optical interference signal to perform frequency analysis for computation in a frequency space (S12). Subsequently, the control computation unit 14 performs bandpass filtering in association with the modulation frequency of the optical mechanism 10 to improve the signal-to-noise ratio (SNR) of the signal (S14), and then performs Hilbert transform (S16). The control computation unit 14 performs an autocorrelation-type real-time viscoelasticity computing process by using an analysis signal obtained by the Hilbert transform (S18). At the same time, the control computation unit 14 performs a tomographic viscoelasticity distribution computing process (S20). The real-time viscoelasticity computing process is a process of computing the viscoelasticity of tissue of the object S in one axial direction (Z direction) substantially in real time and visualizing the viscoelasticity in the process of acquiring the OCT images. The tomographic viscoelasticity distribution computing process is a process of acquiring a plurality of OCT images (two-dimensional images), and tomographically visualizing the viscoelasticity of the tissue on the basis of the OCT images in a post-processing manner.

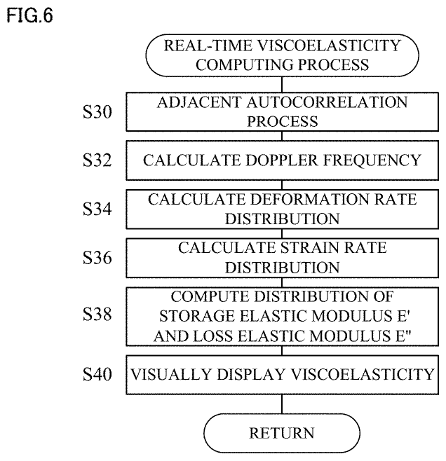

FIG. 6 is a flowchart illustrating the real-time viscoelasticity computing process in S18 of FIG. 5 in detail. The control computation unit 14 performs an adjacent autocorrelation process by using the analysis signal acquired in S16 to obtain a phase difference at certain coordinates (S30) and obtains a Doppler frequency (Doppler angular frequency shift amount) (S32). Subsequently, the control computation unit 14 calculates the tomographic distribution of deformation rate of the tissue by spatial smoothing or spatial averaging (S34). The control computation unit 14 calculates strain distribution (strain rate distribution) on the basis of the deformation rate distribution by using the least-squares method (S36), calculate the distribution of the storage elastic modulus E′ and the loss elastic modulus E″ in the Z direction on the basis of the expression (11) (S38), and tomographically visualizes the viscoelasticity in one axial direction (S40).

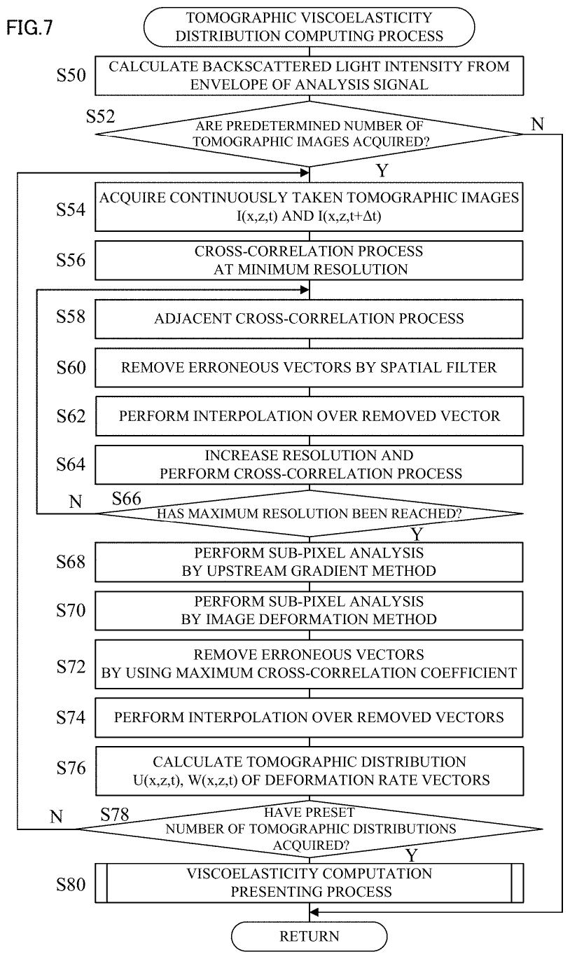

FIG. 7 is a flowchart illustrating the tomographic viscoelasticity distribution computing process in S20 of FIG. 5 in detail. The control computation unit 14 calculates the interference light intensity on the basis of an envelope (amplitude) of the analysis signal (S50). Upon acquiring a predetermined number of OCT images (S52: Y), the control computation unit 14 then reads two continuous tomographic images I(x,z,t) and I(x,z,t+Δt) with different times which are taken continuously (S54). Subsequently, the control computation unit 14 performs processes of the recursive cross-correlation method. Herein, the control computation unit 14 first performs a cross-correlation process at minimum resolution (an interrogation region of a maximum size), and obtains correlation coefficient distributions (S56). Subsequently, the control computation unit 14 computes a product of adjacent correlation coefficient distributions by the adjacent cross-correlation multiplication (S58). At this point, the control computation unit 14 removes erroneous vectors by a spatial filter such as a standard deviation filter (S60), and performs interpolation over the removed vectors by the least-squares method or the like (S62). Subsequently, the control computation unit 14 reduces the interrogation region in size to increase the resolution, and continue the cross-correlation process (S64). Thus, the cross-correlation process is performed on the basis of a reference vector at low resolution. If the resolution at this point has not reached a predetermined maximum resolution (S66: N), the control computation unit 14 returns to S58.

The control computation unit 14 then repeats the processes in S58 to S66, and when the cross-correlation process at the maximum resolution is completed (S66: Y), performs the sub-pixel analysis. Specifically, the control computation unit 14 computes a sub-pixel displacement by the upstream gradient method on the basis of the distribution of deformation vectors at the maximum resolution (the interrogation region of the minimum size) (S68). The control computation unit 14 then computes a sub-pixel deformation quantity by the image deformation method on the basis of the obtained sub-pixel displacement (S70). Subsequently, the control computation unit 14 removes erroneous vectors by filtering using a maximum cross-correlation value (S72), and performs interpolation over the removed vectors by the least-squares method or the like (S74). The control computation unit 14 then differentiates the thus obtained deformation vectors with respect to time to calculate tomographic distribution U(x,z,t), W(x,z,t) of deformation rate vectors of the tomographic images (S76). Note that U(x,z,t) represents a component in the z direction of the deformation rate vector distribution, and W(x,z,t) represents a component in the x direction of the deformation rate vector distribution. The above-described processes are also performed on subsequent tomographic images (S78: N). When the tomographic distributions of the deformation vectors are obtained for a preset number of tomographic images (S78: Y), the control computation unit 14 performs a viscoelasticity computation presenting process (S80). If the preset number of OCT images have not been acquired (S52: N), the control computation unit 14 temporarily terminates the present process.

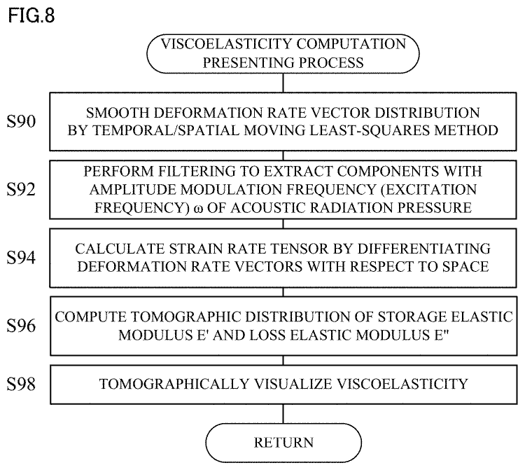

FIG. 8 is a flowchart illustrating the viscoelasticity computation presenting process in S80 of FIG. 7 in detail. The control computation unit 14 performs smoothing on the deformation rate vector distribution calculated in S76 by the temporal/spatial moving least-squares method (S90). The control computation unit 14 then extracts, from the smoothed deformation rate vectors, only components in synchronization with excitation frequency co (amplitude modulation frequency) caused by the acoustic radiation pressure (S92). The control computation unit 14 then differentiates the deformation rate vectors with respect to space to calculate a strain rate tensor (S94).

Subsequently, the control computation unit 14 calculates the strain amount by time integration of the strain rate obtained in S94, and computes tomographic distribution of the amplitude (strain amplitude ε0) of the strain amount. As described above, the control computation unit 14 then computes the tomographic distribution of the storage elastic modulus E′ and the loss elastic modulus E″ by using the amplitude (stress amplitude σ0) of the acoustic radiation pressure of the ultrasonic waves and the phase difference (loss angle δ) between stress and strain (S96), and tomographically visualizes the viscoelasticity of the tissue (S98). Note that formulation is also possible using the strain rate. In other words, the association between the strain rate and the viscoelasticity parameters can be obtained.

Next, verification experiments conducted for evaluation of the effects of the present example will be explained.

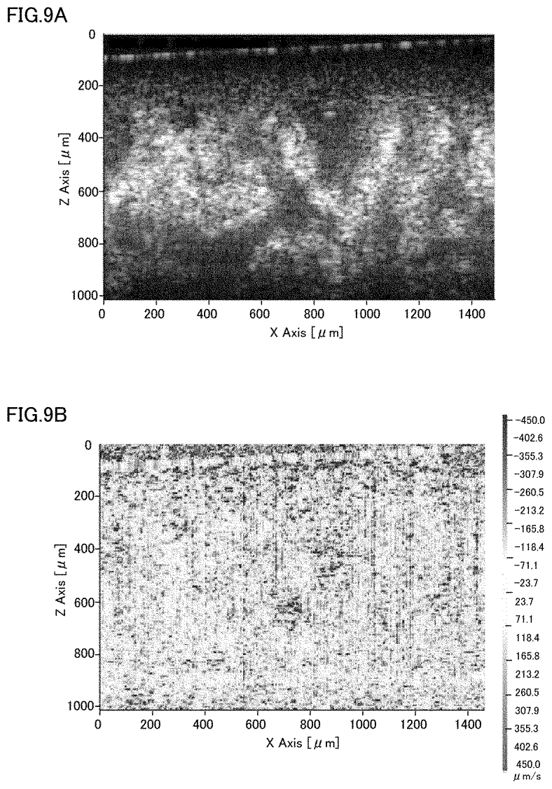

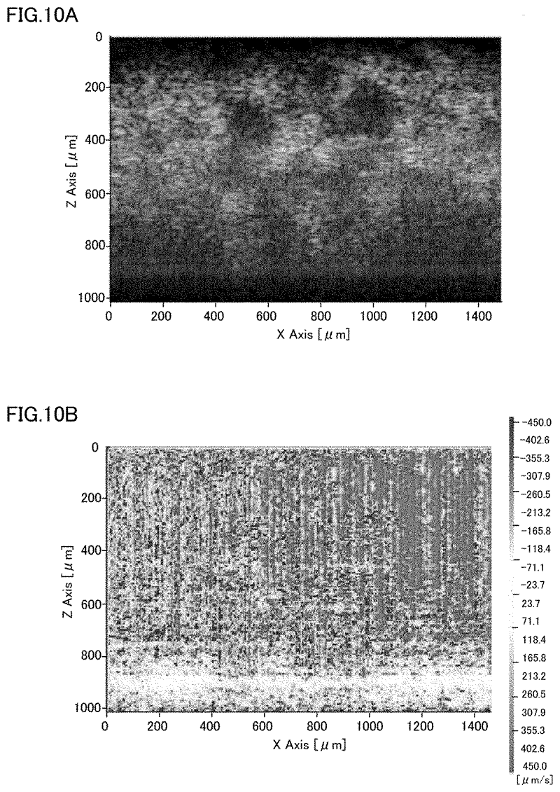

FIGS. 9A, 9B, 10A, and 10B illustrate results of experiments with samples that are imitations of biological tissue. FIGS. 9A and 9B illustrate results in a case where a rigid gel sample was used, and FIGS. 10A and 10B illustrate results in a case where a soft gel sample was used. FIGS. 9A and 10A illustrate OCT images, and FIGS. 9B and 10B illustrate tomographic distributions of deformation rate (refer to the expression (7) above).

In the experiments, an SLD broadband light source with a center wavelength λ_{c}=1317 nm and a full width at half maximum Δλ=100 nm was used as the light source 2. The reference mirror 26 was set to have a deflection angle of ±1.9 degrees, a scanning frequency of 4 kHz, and a depth scanning range (Z-direction detection range) of 900 μm in the samples. The sampling frequency was set to 10 MHz, a single-axis, depth interference signal was acquired at intervals of Δx=2.5 μm, and a tomographic image of 1500×900 (μm) was obtained. In addition, the ultrasound device 72 was driven by a fundamental wave of 1 MHz, and an applied acoustic pressure was measured by a hydrophone before the experiments.

Gel samples (15 mm×15 mm×5 mm) imitating the optical characteristics of human skin were used as the object S. Note that two kinds of gel samples, which are a soft gel sample with 0.5% by weight of gel hardener and a rigid gel sample with four times more gel hardener than the soft gel sample, were prepared, and tomographic visualization was evaluated on rigidity dependence.

The experiment results illustrated in FIGS. 9A, 9B, 10A, and 10B show that the deformation rate is lower in the rigid gel sample having higher rigidity, and that the deformation rate is higher in the soft gel sample having lower rigidity. This can be said to be consistent with the fact that the vibration of low-rigidity part of biological tissue relative to exciting force is great (large amplitude) and that the vibration of high-rigidity part thereof is small (small amplitude). Note that it is considered that the deformation rate of the soft gel sample is distributed because the acoustic radiation pressure is distributed.

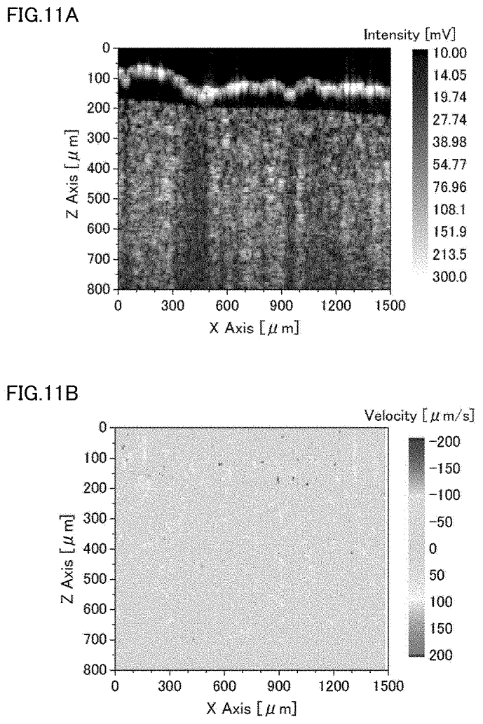

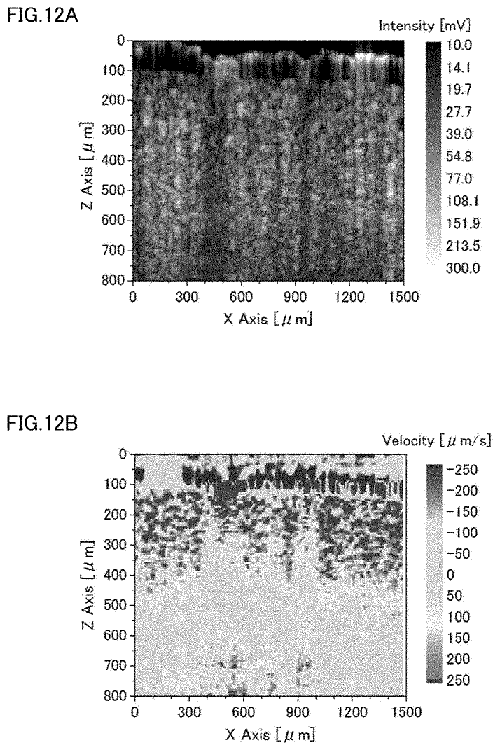

FIGS. 11A, 11B, 12A, and 12B illustrate results of experiments with a layered sample with rigidity varying by the layer. FIGS. 11A and 11B illustrate experiment results in a state in which no acoustic radiation pressure was applied, and FIGS. 12A and 12B illustrate experiment results in a state in which acoustic radiation pressure was applied. FIGS. 11A and 12A illustrate OCT images, and FIGS. 11B and 12B illustrate tomographic distributions of deformation rate. A layered sample including a soft gel layer from the surface to a depth of about 200 μm and a rigid gel layer from the depth of about 200 μm to about 1000 μm was used.

The experiment results illustrated in FIGS. 11A and 11B show that no difference was observed between the respective layers of the sample in the state in which no acoustic radiation pressure was loaded. In contrast, the experiment results illustrated in FIGS. 12A and 12B show that the deformation rate is different between the soft gel layer and the rigid gel layer (the deformation rate (amplitude) is higher in the soft gel layer). This conversely means that the tomographic distribution of the rigidity in an object (tomographic distribution of E′ in distribution of elasticity, that is, the expression (11) above) can be evaluated by tomographic visualization of the deformation rate.

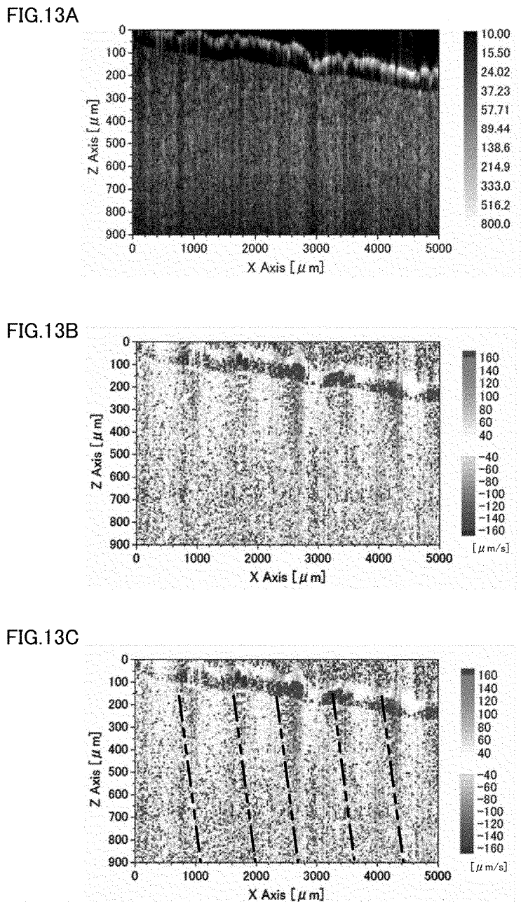

FIGS. 13A to 13C illustrate results of experiment on viscosity evaluation. FIG. 13A illustrates an OCT image, and FIG. 13B illustrates tomographic distribution of deformation rate. FIG. 13C illustrates that a phase lag of the deformation rate in the depth direction (Z direction) is present in FIG. 13B by alternate long and two short dashes lines.

In this experiment, the same sample as that illustrated in FIGS. 12A and 12B was used. The amplitude of acoustic radiation pressure caused by ultrasonic waves was changed periodically while scanning in the X direction was performed by the OCT, and the obtained deformation rate was tomographically visualized. In this experiment, the amplitude modulation frequency was 2.4 Hz.

The experiment result shows that the phase of the deformation rate is delayed toward the depth direction (the phase is shifted between the upper layer and the lower layer) (the phase is indicated by the alternate long and two short dashes lines). This shows that a phase difference (loss angle δ) is present between stress loaded on the sample by acoustic radiation pressure and deformation (strain) caused by the stress, and that the sample is viscous. Thus, the results of experiment shows that the tomographic distribution of viscosity (tomographic distribution of E″ in the distribution of viscosity coefficient, that is the expression (11) above) can be evaluated. It is suggested that the viscoelasticity of biological tissue can be tomographically detected by the example above.

As described above, according to the present example, the viscoelasticity of the tissue of an object S is tomographically visualized in microscale by non-contact load application by acoustic radiation pressure and non-contact and non-invasive tomographic measurement by the OCT. This prevents contamination of the object S and enables evaluation of the mechanical characteristics thereof with high accuracy even when the object S requires strict quality assurance. This is considered to contribute to solution to biomechanics of regenerated tissue.

The description of the present invention given above is based on illustrative examples. It will be obvious to those skilled in the art that the present invention is not limited to the particular examples but various modifications could be further developed within the technical idea underlying the present invention.

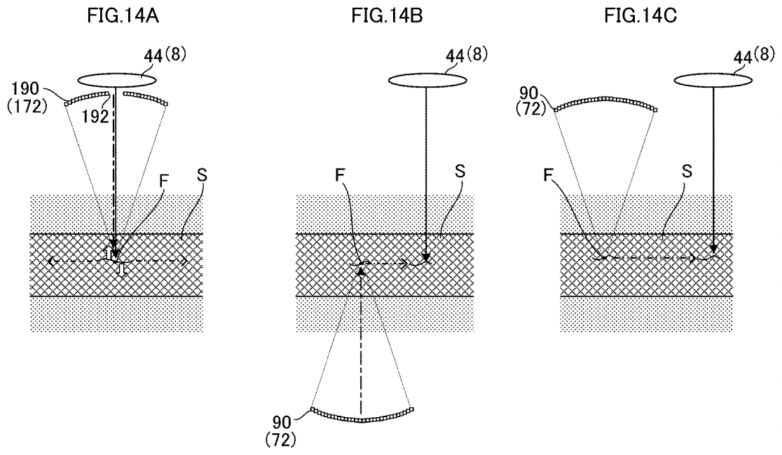

FIGS. 14A to 14C are diagrams illustrating load application methods according to modifications. FIG. 14A illustrates a first modification, FIG. 14B illustrates a second modification, and FIG. 14C illustrates a third modification.

In the first modification, the optical mechanism 8 and an ultrasound device 172 are on the same side with respect to an object S, and the optical axis of the OCT is set to pass through the focus point F. In this configuration, a transducer array 190 is formed in a donut shape with a hole 192 at the center. Light of the OCT is directed to the object S through the hole 192. According to such a configuration, the support of the loading device need not have the through-hole as in the example described above.

In the second modification, the optical mechanism 8 and the ultrasound device 72 are arranged on the sides opposite to each other with respect to an object S, and the optical axis does not pass through the focus point F in some cases in the process of tomographic measurement by the OCT. For example, a case where the focus point F of the ultrasound device 72 is fixed and scanning in the X direction is performed by the OCT corresponds to such cases. In this modification, deformation of tissue at a position at a predetermined distance from the focus point F in the X direction is detected. In the third modification, however, the optical mechanism 8 and the ultrasound device 72 are arranged on the same side with respect to an object S, and the optical axis is set is set not to pass through the focus point F in the process of tomographic measurement by the OCT.

Setting the optical axis of the OCT to pass through the focus point F as in the example described above and the first modification facilitates recognition of the amplitude and the phase of the acoustic radiation pressure at a position of detection by the OCT, which facilitates the computations and also facilitates an increase in the accuracy of the computations. In view of the space for installation of the mechanisms and devices and the space for operation thereof, the second and third modifications are more advantageous in that the system can be easily built, or the like.

Note that the configurations according to the second and third modifications may be used to detect the propagation speed of a shear wave instead of the deformation of tissue by acoustic radiation pressure. The viscoelasticity of tissue may then be determined on the basis of the propagation speed. In other words, non-contact elastography using acoustic radiation pressure may be used to detect the viscoelasticity of tissue by the OCT.

In the example and modifications described above, examples in which the support 70 is fixed and the optical axis of the OCT and the focus point of the ultrasonic waves are shifted have been presented. In a modification, the support 70 may be controlled to move in the X direction and the Y direction, so that the focus point of ultrasonic waves on an object S and position of detection by the OCT are changed.

In the example described above, an example in which an object to be measured is regenerated tissue (cultured cells) has been presented. In a modification, the device may be applied to a regenerative therapy of a living body to monitor the engraftment capacity of tissue. The device is applicable not only to the medical field such as cartilage diagnosis and skin diagnosis, but also to various uses and fields such as the fields of cosmetic surgery and cosmetic industry.

In the example described above, an example in which the ultrasound device is employed as a non-contact loading device and tissue deformation is induced by acoustic radiation pressure has been presented. In a modification, electrically conductive or magnetic particles (such as molecular target drug) may be introduced into biological tissue, and the tissue may be irradiated with an electromagnetic field, so that tissue deformation is induced.

While an example in which viscoelasticity evaluation is performed on the basis of the storage elastic modulus E′ and the loss elastic modulus E″ has been presented in the example above, a value expressing a physical value of viscoelasticity may be newly defined from the storage elastic modulus E′ and the loss elastic modulus E″, and viscoelasticity evaluation may be performed on the basis of the physical value.

While an example in which tomographic images are acquired two-dimensionally by the OCT has been presented in the example above, tomographic images may be acquired three-dimensionally. Specifically, scanning may be carried out not only in the depth direction (Z direction) and the X direction but also in the Y direction for tomographic visualization of the viscoelasticity of tissue.

The present invention is not limited to the above-described examples and modifications only, and the components may be further modified to arrive at various other examples without departing from the scope of the invention. Various other examples may be further achieved by combining, as appropriate, a plurality of components disclosed in the above-described example and modifications. Furthermore, one or some of all of the components exemplified in the above-described example and modifications may be left unused or removed.

What is claimed is:

1. A viscoelasticity visualizing device that includes an optical system using optical coherence tomography, and tomographically visualizes viscoelasticity of tissue of an object to be measured, the viscoelasticity visualizing device comprising:
an optical mechanism, the optical mechanism guiding light from a light source to the tissue to scan the tissue;
a loading device that applies deformation energy to the tissue;
a control computation unit that computes tomographic distribution of viscoelasticity of the tissue by controlling driving of the loading device and the optical mechanism, and processing an optical interference signal from the optical system on the basis of the driving; and
a display device that displays the viscoelasticity of the tissue in a form of tomographic visualization, wherein
the loading device is a non-contact loading device that loads acoustic radiation pressure in the tissue by outputting ultrasonic waves to a focus point set in the object.

2. The viscoelasticity visualizing device according to claim 1, wherein an axis of light emitted toward the object is set to pass through the focus point of the acoustic radiation pressure in the object.

3. The viscoelasticity visualizing device according to claim 1, wherein the control computation unit computes a predetermined mechanical feature quantity caused by deformation of the tissue or a change in a predetermined mechanical feature quantity accompanying the deformation of the tissue in association with a cross-sectional position in the object, and computes tomographic distribution of the viscoelasticity of the tissue on the basis of results of the computation.

4. The viscoelasticity visualizing device according to claim 1, wherein
the optical mechanism emits light into the object through a front side of the object,
the loading device outputs ultrasonic waves into the object through a rear side of the object.

5. The viscoelasticity visualizing device according to claim 4, further comprising:
a support that supports a translucent and transaudient container for accommodating regenerated tissue as the object, wherein
the support has a hole through which the rear side of the container is exposed.

6. The viscoelasticity visualizing device according to claim 1, wherein
the optical mechanism comprises:
a first optical mechanism, the first optical mechanism being provided on an object arm that includes the object, the first optical mechanism guiding light from the light source to the object to scan the object; and
a second optical mechanism, the second optical mechanism being provided on a reference arm that does not include the object, the second optical mechanism guiding light from the light source to a reference mirror, so that the light is reflected by the reference mirror,
the viscoelasticity visualizing device further comprises an optical detector that detects interference light resulting from superimposition of object light reflected by the object and reference light reflected by the reference mirror on each other, and
the control computation unit performs frequency analysis on the optical interference signal input from the optical detector, calculates a deformation rate of the tissue by using a Doppler frequency, and computes the viscoelasticity of the tissue on the basis of strain information and acoustic radiation pressure information from the loading device, the strain information being obtained on the basis of the deformation rate.

7. The viscoelasticity visualizing device according to claim 6, wherein the control computation unit computes the viscoelasticity of the tissue on the basis of a phase difference between a phase of exciting force caused by the acoustic radiation pressure and a phase of the strain.

8. A viscoelasticity visualizing method for tomographically visualizing viscoelasticity of tissue of an object to be measured, the viscoelasticity visualizing method comprising:
a loading step of applying a load into the tissue by acoustic radiation pressure of ultrasonic waves;
an interference signal acquiring step of acquiring, by using optical coherence tomography, an optical interference signal based on backscattered light from the tissue that is deformed by the application of the load;
a computing step of processing the optical interference signal and computing tomographic distribution of the viscoelasticity of the tissue on the basis of a change in a predetermined mechanical feature quantity accompanying deformation of the tissue; and
a displaying step of tomographically visualizing the viscoelasticity of the tissue.
